(19) United States
(12) Patent Application Publication
(10) Pub. No.: US 2023/0159433 A1
(43) Pub. Date: May 25, 2023

(54) METHOD FOR PREPARING TOLUYLENE DIAMINE MIXTURES

(71) Applicant: Covestro Deutschland AG, Leverkusen (DE)

(72) Inventors: Sebastian Pohl, Leverkusen (DE); Bernd Pennemann, Bergisch Gladbach (DE)

(21) Appl. No.: 17/997,250

(22) PCT Filed: May 18, 2021
(86) PCT No.: PCT/EP2021/063093

§ 371 Date: Oct. 27, 2022

(30) Foreign Application Priority Data
May 19, 2020 (EP) 20175311.8
Mar. 22, 2021 (EP) 21163899.4

(51) Int. Cl. C07C 209/88

(52) U.S. Cl. CPC C07C 209/88

(57) ABSTRACT

The invention relates to a method for preparing a toluylene diamine mixture which, along with toluylene diamine (TDA), also contains a high-boiling fraction, such as the high-boiling fraction which is accumulated as a sump flow in the distillative preparation of product mixtures obtained by hydrogenating dinitrotoluene. The method has a step (A), namely preparing a TDA mixture containing, based on the total mass of the mixture, (1) TDA in a range of 5 mass % to 80 mass % and (2) a high-boiling fraction in a range of 20 mass % to 95 mass %; a step (B), namely distilling TDA off from the TDA mixture, thereby obtaining a liquid TDA-depleted method product, containing (1) TDA in a range of 0 mass % to 38 mass % and (2) a high-boiling fraction in a range of 62 mass % to 100 mass %; and a step (C) namely mixing water into the TDA-depleted method product in a mixing chamber, thereby obtaining a mixture mixed with water, wherein the temperature and quantity of the water to be mixed into the mixture and the temperature and quantity of the TDA-depleted method product are matched such that the resulting temperature of the mixture mixed with water ranges from 110° C. to 250° C., and the mixture mixed with water is provided as a single phase. The mixing chamber is supplied with a pressure which is greater than or equal to the water vapor partial pressure at the resulting temperature.

The present invention relates to a process for workup of a tolylenediamine mixture which contains not only tolylenediamine (TDA) but also high boilers, as obtained in particular as a bottoms stream from the distillative workup of product mixtures obtained by hydrogenation of dinitrotoluene. The process comprises a step (A), namely providing a TDA mixture containing, based on its total mass, (1) TDA in a range from 5% by mass 80% by mass and (2) high boilers in a range from 20% by mass to 95% by mass; a step (B), namely distillative removal of TDA from the TDA mixture to obtain a liquid process product depleted in TDA, containing (1) TDA in a range from 0% by mass to 38% by mass and (2) high boilers in the range from 62% by mass to 100% by mass; and a step (C), namely incorporation of water into the process product depleted in TDA in a mixing space to obtain a mixture admixed with water, wherein the temperature and amount of the water to be incorporated and the temperature and amount of the process product depleted in TDA are matched to one another such that a temperature of the mixture admixed with water in the range from 110° C. to 250° C. results and the mixture admixed with water is monophasic, wherein a pressure which is greater than or equal to the water vapor partial pressure at the resulting temperature is established in the mixing space.

Tolylenediamine (TDA) is an important intermediate in the production of the isocyanate tolylene diisocyanate (TDI) which has a wide range of applications, in particular in the production of flexible foams, for which the meta-isomers (2,4- and 2,6-TDA) are of particular interest. But also the orthoisomers (2,3-TDA and 3,4-TDA) have industrial applications (for example as starters in polyether polyol production).

TDA is typically obtained by catalytic hydrogenation of dinitrotoluene (DNT). This initially affords a water-containing crude product which contains not only the target product TDA but also organic impurities having higher and lower boiling points than said product (so-called high boilers and low boilers respectively). Initially water is largely removed from this crude product. This is followed by a distillative workup comprising an isomer separation and purification for separation of the high boilers and low boilers. This distillative workup affords a bottoms fraction which contains high boilers separated from the target product TDA and also proportions of TDA. It is in principle sought to keep the proportion of TDA in this bottoms function as low as possible in order, ideally, not to lose any valuable product. On the other hand it is essential for distillation on an industrial scale that the bottoms fraction of a distillation column does not become too viscous and in particular remains liquid over the entire operating range. It is therefore customary in practice to leave a certain proportion of TDA in the high boiler-containing bottoms fraction in the distillative purification of the crude dewatered TDA. The remaining, high-boiling constituents of the bottoms fraction are often referred to as distillation residue or residue for short.

U.S. Pat. No. 6,359,177 B1 describes a process for distillation of mixtures such as especially TDA isomer mixtures and a distillation assembly suitable for performing the process. The process has the feature that it affords not only a distillate fraction rich in meta-TDA (streams 4, 40, 41) but also a bottoms fraction rich in high boilers and ortho-TDA (streams 5, 50, 51). It is unclear from this document how ortho-TDA, which certainly has applications as mentioned above, can be advantageously recovered from the bottoms fraction without the remaining high boilers solidifying.

EP 1 746 083 A1 describes the distillative purification of crude TDA using a dividing wall column. A meta-TDA-rich distillate fraction is obtained as a side draw from the dividing wall column while a high boiler-rich stream which contains meta-TDA in addition to the high boilers is obtained in the bottom of said column. In one variant of this process the high boiler-rich stream discharged from the bottom of the dividing wall column is subjected to further processing to reduce the losses of meta-TDA. This may be effected using a kneader-dryer for example. Said kneader-dryer is operated with heating under vacuum. The meta-TDA obtained in the feed to the kneader-dryer is thus evaporated. The vapor stream thus obtained may be supplied to a condenser for example to obtain meta TDA which may be recycled into the process or mixed with the meta-TDA product stream. The vapor stream can also be recycled directly into the dividing wall column. In order to reduce the viscosity of the high boiler-rich process product obtained in the kneader-dryer, said product may be mixed with a suitable low-viscosity liquid, such as in particular with a low-boiler stream obtained in the distillation of the TDA and/or with an ortho-TDA-containing product stream obtained in the distillation of the TDA. Since the low-boiler stream likewise contains proportions of ortho-TDA, this process does not manage to avoid a loss of TDA isomers either.

WO 2018/140461 A1 describes a process for reducing the solidification point of a TDA residue composition, as obtained as bottoms product in the distillative workup of TDA, by addition of low-viscosity and low-boiling substances such as especially water. The aim is in particular to make it easier to use the TDA residue composition as fuel. Addition of process water at a temperature of 40° C. to such a TDA residue composition temperature heated to slightly below 100° C. in proportions of 10% to 20% by mass made it possible to achieve a significant viscosity reduction (cf. example 4 and table 1). TDA residue compositions, into which water is incorporated, are compositions containing TDA isomers such as meta-TDA, ortho-TDA or mixtures of both in mass fractions of 40% to 75% and residue in mass fractions of 25% to 60% (see for example paragraph [0028]). In this process too the TDA remains in the TDA residue composition/in the mixture obtained therefrom by addition of water. It is unclear from this document how TDA can be recovered from the TDA residue composition containing TDA and mass fractions of 40% to 75%, i.e. how the TDA content of the TDA residue composition can be reduced to values of less than 40% by mass, without the remaining high-boilers solidifying or forming two phases.

EP 0 705 814 A1 relates to a process for producing a meta-TDA isomer mixture for storage and transport, especially longer-term storage or storage in large containers, for example shipping containers. In this process a mixture of 2,4- and 2,6-TDA (=meta-TDA) is distilled under reduced pressure and at elevated temperatures in order to obtain, by separation of water, low boilers, ortho-TDA (which has a lower boiling point than meta-TDA) and other secondary components, a substantially water-free product which is rich in meta-TDA (mixture of 2,4-TDA and 2,6-TDA). However, this product fraction is obtained as the bottoms stream from the distillation (cf. column 1, lines 24 and 25) which means that the described distillation does not separate high-boiling secondary components (high boilers) which make up the distillation residue. Distillation of TDA before high-boiler separation is thus described. The resulting meta-TDA therefore contains high boilers but only in small proportions because concentration of the high boilers, as in the bottoms fraction of a high boiler separation (in which the target product meta-TDA is the distillate stream), has not yet taken place. In any case it may be assumed that the mass fraction of high boilers in the substantially water-free meta-TDA described is low. According to the abovementioned US patent U.S. Pat. No. 6,359,177 B1 regarding the composition of various TDA fractions in the TDA distillation, a residue fraction in the lower single-digit percentage range may be expected. The process has the feature that the melting point of the resulting meta-TDA is reduced by incorporating water in an amount of 5% to 15% by weight into a melt of the meta-TDA and adjusting the temperature of the resulting meta-TDA mixture such that its end temperature is at or below the boiling point. The meta-TDA mixture treated in this way may be stored or transported. This document is therefore not at all concerned with the treatment of distillation residuesor mixtures of TDA and substantial proportions of distillation residues.

There was therefore a need for further improvements in the field of distillative workup of TDA. It would especially be desirable to minimize the proportion of TDA isomers, which are left in high boiler-rich fractions (such as for example the TDA residue composition described in WO 2018/140461 A1) merely for the purpose of viscosity reduction, without impairing the processability of the remaining high boilers due to an excessive viscosity increase.

Taking account of this need the present invention therefore provides a process for workup of a tolylenediamine mixture containing tolylenediamine and compounds having boiling points higher than 2,4-tolylenediamine at 1013 mbar_{(abs.)} (high boilers) (such as is obtained especially as a bottoms stream from the distillative workup of a product mixture obtained by catalytic hydrogenation of dinitrotoluene), comprising the steps of:
(A) providing a tolylenediamine mixture containing, based on its total mass, (1) tolylenediamine in a range from 5% by mass to 80% by mass and (2) compounds having boiling points higher than 2,4-tolylenediamine at 1013 mbar(_{abs}.) (high boilers) in a range from 20% by mass to 95% by mass;
(B) distillative removal of tolylenediamine from the tolylenediamine mixture (i.e. the tolylenediamine mixture provided in (A) containing, based on its total mass, (1) tolylenediamine in a range from 5% by mass to 80% by mass and (2) compounds having boiling points higher than 2,4-tolylenediamine at 1013 mbar(_{abs}.) in a range from 20% by mass to 95% by mass) to obtain a liquid process product depleted in tolylenediamine containing (1) tolylenediamine in a range from 0% by mass (in particular 0.1% by mass) to 38% by mass (preferably 1% by mass to 37% by mass, particularly preferably 15% by mass to 36% by mass) and (2) compounds having boiling points higher than 2,4-tolylenediamine at 1013 mbar(_{abs}.) (high boilers) in a range from 62% by mass to 100% by mass (in particular 99.9% by mass) (preferably 63% by mass to 99% by mass, particularly preferably 64% by mass 85% by mass);
C) incorporating water into the process product depleted in tolylenediamine (i.e. the liquid process product depleted in tolylenediamine containing (1) tolylenediamine in a range from 0% by mass (in particular 0.1% by mass) to 38% by mass (preferably 1% by mass to 37% by mass, particularly preferably 15% by mass to 36% by mass) and (2) compounds having boiling points higher than 2,4-tolylenediamine at 1013 mbar(_{abs}.) (high boilers) in a range from 62% by mass to 100% by mass (in particular 99.9% by mass) (preferably 63% by mass to 99% by mass, particularly preferably 64% by mass 85% by mass) obtained in (B)) in a mixing space to obtain a mixture admixed with water, wherein the temperature and amount of the water to be incorporated and the temperature and amount of the process product depleted in tolylenediamine are matched to one another such that a temperature of the mixture admixed with water in the range from 110° C. to 250° C. results and the mixture admixed with water is monophasic, wherein a pressure (henceforth also pressure in the mixing space) which is greater than or equal to the water vapor partial pressure at the resulting temperature (=the temperature of the mixture admixed with water vapor) is established in the mixing space.

In the context of the present invention high boilers are to be understood as meaning organic compounds having boiling points at 1013 mbar_{(abs.)} higher than that of the highest boiling TDA isomer, 2,4-tolylenediamine. The high boilers are therefore organic secondary components distinct from the TDA isomers. Low boilers are to be understood as meaning organic compounds distinct from the TDA isomers having boiling points at 1013 mbar_{(abs.)} lower than that of 2,4-tolylenediamine.

Pressures are reported here and in the following as absolute pressures indicated by a subscript “(abs.)” after the units of pressure (generally mbar).

The requirement according to the invention that the mixture admixed with water obtained in step (C) is monophasic is to be understood as meaning that a mixture admixed with water that is flowable at the conditions of pressure and temperature selected for step (C) is formed and, after initial mixing, even at rest (i.e. without further mechanical mixing by a pump or a stirrer) does not undergo any discernible formation of a further phase (precipitation of solid or formation of a further liquid phase, for instance as a continuous phase of coalescing droplets) in industrially relevant timeframes (i.e. at least 1 h, preferably 1 h to 720 h, particularly preferably 24 h to 480 h, very particularly preferably 48 h to 360 h).

The water vapor partial pressure at the resulting temperature is obtainable for example from the materials database DETHERM which is known in the art or the technical literature, in particular from VDI-Wärmeatlas 2013 (DOI 10.1007/978-3-642-19981-3_12), chapter D2, Stoffwerte von bedeutenden reinen Fluiden, subchapter D2.1, Wasser, pages 175 to 195, in particular table 2 (values for pₛ).

There follows firstly a brief summary of various possible embodiments.

In a first embodiment of the process according to the invention, which may be combined with all other embodiments, in step (C) a temperature of the mixture admixed with water in the range from 120° C. to 220° C. is established.

In a second embodiment of the process according to the invention, which is a particular configuration of the first embodiment, in step (C) a temperature of the mixture admixed with water in the range from 120° C. to 160° C. is established.

In a third embodiment of the process according to the invention, which may be combined with all other embodiments, in step (C) the pressure in the mixing space is established by introducing a gas selected from the group consisting of air, nitrogen, carbon dioxide, helium and argon.

In a fourth embodiment of the process according to the invention, which may be combined with all other embodiments, but which may also be used as an alternative to the third embodiment, in step (C) the pressure in the space is established via a pressure control valve.

In a fifth embodiment of the process according to the invention, which may be combined with all other embodiments, the mass of the water incorporated in step (C) corresponds to 10% to 100% of the mass of the tolylenediamine distilled off in step (B).

In a sixth embodiment of the process according to the invention, which is a particular configuration of the fifth embodiment, the mass of the water incorporated in step (C) corresponds to 10% to 80% of the mass of the tolylenediamine distilled off in step (B).

In a seventh embodiment of the process according to the invention, which may be combined with all other embodiments, the water to be incorporated into the process product depleted in tolylenediamine in step (C) has a temperature in the range from 10° C. to 100° C.

In an eighth embodiment of the process according to the invention, which is a particular configuration of the seventh embodiment, the water to be incorporated into the process product depleted in tolylenediamine in step (C) has a temperature in the range from 20° C. to 90° C.

In a ninth embodiment of the process according to the invention, which is a particular configuration of the seventh embodiment, the water to be incorporated into the process product depleted in tolylenediamine in step (C) has a temperature in the range from 10° C. to 40° C. (so that fresh water or low-temperature condensate is a suitable water source) or in the range from 80° C. to 100° C. (so that high-temperature condensate is a suitable water source).

In a tenth embodiment of the process according to the invention, which is a particular configuration of the eighth embodiment, the water to be incorporated into the process product depleted in tolylenediamine in step (C) has a temperature in the range from 20° C. to 40° C. (so that fresh water or low-temperature condensate is a suitable water source) or in the range from 80° C. to 90° C. (so that high-temperature condensate is a suitable water source).

In an eleventh embodiment of the process according to the invention, which may be combined with all other embodiments, step (C) is followed by the following steps:
(D.I) preparing the mixture admixed with water for a use as material (i.e. as a starting material for obtaining other products) or for a thermal use (i.e. as a fuel) comprising decompressing and cooling the mixture admixed with water to a temperature in the range from 20° C. to 100° C. to obtain a decompressed and cooled mixture admixed with water;
(E) supplying the decompressed and cooled mixture admixed with water obtained in step (D) to a use as material or thermal use.

In a twelfth embodiment process according to the invention, which is a particular configuration of the eleventh embodiment, the cooling in step (D.I) is carried out to a temperature in the range from 20° C. to 90° C.

In a thirteenth embodiment of the process according to the invention, which may be combined with all embodiments comprising steps (D.I) and (E), the decompressing in step (D.I) is carried out to ambient pressure.

In a fourteenth embodiment of the process according to the invention, which may be combined with all embodiments comprising steps (D.I) and (E) the decompressed and cooled mixture admixed with water is, prior to its use as material or thermal use in step (E), stored and/or transported at a temperature in the range from ambient temperature to the temperature to which it was cooled in step (D.I), wherein the storing and transporting altogether account for a period of 1 h to 720 h.

In a fifteenth embodiment of the process according to the invention, which is a particular configuration of the fourteenth embodiment, the storing and transporting altogether account for a period of 24 h to 480 h.

In a sixteenth embodiment of the process according to the invention, which is a particular configuration of the fifteenth embodiment, the storing and transporting altogether account for a period of 48 h to 360 h.

In a seventeenth embodiment of the process according to the invention, which may be combined with all embodiments which comprise steps (D.I) and (E) and do not provide for a use as material, the use in step (E) is a thermal use.

In an eighteenth embodiment of the process according to the invention, which may be combined with all embodiments which comprise steps (D.I) and (E) and do not provide for a thermal use, the use in step (E) is a use as material which comprises the use of the mixture admixed with water as a feedstock in a pyrolysis for recovery of benzene, toluene and/or xylene.

In a nineteenth embodiment of the process according to the invention, which can be combined with all embodiments which do not provide for steps (D.I) and (E), step (C) is followed by the following step:
(D.II) thermal use of the mixture mixed with water (i.e. using the mixture admixed with water as fuel).

In a twentieth embodiment of the process according to the invention, which may be combined with all other embodiments, the tolylenediamine mixture in step (A) is provided by distillative workup of a meta-tolylenediamine-containing tolylenediamine isomer mixture, wherein a (first) meta-tolylenediamine product fraction is obtained in addition to the tolylenediamine mixture (provided in step (A)).

In a twenty-first embodiment of the process according to the invention, which is a particular configuration of the twentieth embodiment, the distillative workup comprises a distillation in a distillation column (in particular without a dividing wall) with an evaporator arranged downstream thereof, wherein the tolylenediamine mixture (provided in step (A)) is withdrawn from a (first) liquid bottoms stream from the evaporator or from a (second) liquid bottoms stream obtained by further workup thereof and the (first) meta-tolylenediamine product fraction is obtained as a distillate fraction from the evaporator.

In a twenty-second embodiment of the process according to the invention, which is a further particular configuration of the twentieth embodiment, the distillative workup comprises a distillation in a distillation column (in particular without a dividing wall) with an evaporator arranged upstream thereof, wherein the tolylenediamine mixture is withdrawn from a (first) liquid bottoms stream from the evaporator or from a (second) liquid bottoms stream obtained by further workup thereof and the (first) meta-tolylenediamine product fraction is obtained as a liquid bottoms fraction from the distillation column.

In a twenty-first embodiment of the process according to the invention, which is a further particular configuration of the twentieth embodiment, the distillative workup comprises a distillation in a distillation column (in particular without a dividing wall), wherein the tolylenediamine mixture is withdrawn from a (first) liquid bottoms stream from the distillation column or from a (second) liquid bottoms stream obtained by further workup thereof and the (first) meta-tolylenediamine product fraction is obtained as a gaseous bottoms fraction from the distillation column.

In a twenty-fourth embodiment of the process according to the invention, which is a further particular configuration of the twentieth embodiment, the distillative workup comprises a distillation in a dividing wall column, wherein the tolylenediamine mixture (provided in step (A)) is withdrawn from a (first) liquid bottoms stream from the dividing wall column or from a (second) liquid bottoms stream obtained by further workup thereof and the (first) meta-tolylenediamine product fraction is obtained as a side draw fraction from the dividing wall column.

In a twenty-fifth embodiment of the process according to the invention, which is a particular configuration of one or more of the twentieth to twenty-third embodiments, the tolylenediamine mixture (provided in step (A)) is withdrawn from a (second) liquid bottoms stream obtained by further workup of the (first) liquid bottoms stream from the evaporator/the distillation column, wherein this further workup comprises a stripping and a concentrating with addition of an ortho-tolylenediamine fraction distilled off in the distillation column, so that the tolylenediamine mixture (provided in step (A)) preferably contains ortho-tolylenediamine in a proportion of 30% by mass to 70% by mass based on its total mass.

In a twenty-sixth embodiment of the process according to the invention, which is a particular configuration of the twenty-fifth embodiment, step (B) is performed in an evaporation apparatus selected from the group consisting of kneader-dryers, fluidized bed dryers, roller-dryers, falling film evaporators, thin film evaporators, short path evaporators, screw dryers, natural circulation evaporators, forced circulation evaporators and decompression evaporators.

In a twenty-seventh embodiment of the process according to the invention, which is a particular configuration of the twenty-fifth or twenty-sixth embodiment, the distillative removal in step (B) is performed at a temperature in the range from 60° C. to 240° C. and at a pressure in the range from 0.1 mbar_{(abs.)} to 1013 mbar_{(abs.)}, preferably at a temperature in the range from 120° C. to 240° C. and at a pressure in the range from 0.1 mbar_{(abs.)} to 200 mbar_{(abs.)}.

In a twenty-eighth embodiment of the process according to the invention, which is a particular configuration of one or more of the twenty-fifth to twenty-seventh embodiments, the tolylenediamine distilled off in step (B) (which in the present embodiment consists predominantly of ortho-tolylenediamine) is used as a starter for polyether polyol production, as a dye additive, as a starting material for the production of corrosion inhibitors or in aminolysis reactions (in particular for cleavage of urethane bonds in processes for recycling polyurethane products).

In a twenty-ninth embodiment of the process according to the invention, which is a particular configuration of one or more of the twentieth to twenty-fourth embodiments, the tolylenediamine mixture is withdrawn from the (first) liquid bottoms stream from the evaporator/the distillation column/the dividing wall column, wherein the tolylenediamine mixture (provided in step (A)) preferably contains meta-tolylenediamine in a proportion of 10% by mass to 75% by mass based on its total mass.

In a thirtieth embodiment of the process according to the invention, which is a particular configuration of the twenty-ninth embodiment, in step (C), in addition to water, an amount of tolylenediamine smaller than that distilled off in step (B) is re-added, wherein the ratio of the tolylenediamine isomers to one another in the tolylenediamine distilled off on the one hand and in the tolylenediamine re-added on the other hand are selected such that molar ratio of ortho-tolylenediamine to meta-tolylenediamine in the mixture admixed with water is increased relative to the process product depleted in tolylenediamine.

In a thirty-first embodiment of the process according to the invention, which is a particular configuration of the twenty-ninth or thirtieth embodiment, step (B) is performed in an evaporation apparatus selected from the group consisting of kneader-dryers, fluidized bed dryers, roller-dryers, falling film evaporators, thin film evaporators, short path evaporators, screw dryers, natural circulation evaporators, forced circulation evaporators and decompression evaporators.

In a thirty-second embodiment of the process according to the invention, which is a particular configuration of one or more of the twenty-ninth to thirty-first embodiments, the distillative removal in step (B) is performed at a temperature in the range from 98° C. to 270° C. and at a pressure in the range from 0.7 mbar_{(abs.)} to 730 mbar_{(abs.)}, preferably at a temperature in the range from 110° C. to 250° C. and at a pressure in the range from 2 mbar_{(abs.)} to 300 mbar_{(abs.)}.

In a thirty-third embodiment of the process according to the invention, which is a particular configuration of one or more of the twenty-ninth to thirty-second embodiments, the tolylenediamine distilled off in step (B) (which in the present embodiment consists predominantly of meta-tolylenediamine) is phosgenated to afford tolylene diisocyanate, wherein a (second) meta-tolylene diisocyanate product fraction (which may be obtained together with the first meta-tolylene diisocyanate product fraction) is obtained by subsequent distillative workup.

In a thirty-fourth embodiment of the process according to the invention, which is a particular configuration of one or more of the twentieth to thirty-third embodiments, step (A) comprises the following:
(A.I) catalytic hydrogenation of dinitrotoluene, optionally in the presence of a solvent, to obtain, optionally after removal of the solvent, a crude product fraction of tolylenediamine which contains not only isomers of tolylenediamine but also organic impurities.
(A.II) separation of water from the crude product fraction to obtain the meta-tolylenediamine-containing tolylenediamine isomer mixture;
(A.III) distillative workup of the meta-tolylenediamine-containing tolylenediamine isomer mixture to obtain the (first) meta-tolylenediamine product fraction and the tolylenediamine mixture.

In a thirty-fifth embodiment of the process according to the invention, which is a particular configuration of one or more of the twentieth to thirty-fourth embodiments, the (first) meta-tolylenediamine product fraction is phosgenated and a (first) meta-tolylene diisocyanate product fraction is obtained by subsequent distillative workup.

The embodiments briefly outlined above and further possible configurations of the invention shall be more particularly elucidated hereinbelow. The embodiments may be combined with one another as desired, unless the opposite is apparent from the context.

The toluylenediamine mixture to be provided in step (A) is preferably a mixture such as is obtained as the bottoms stream from a distillation step in the distillative workup of a meta-tolylenediamine-containing tolylenediamine isomer mixture. Such a distillative workup affords not only the tolylenediamine mixture but also a (first) meta-tolylenediamine product fraction. The meta-tolylenediamine-containing tolylenediamine isomer mixture is in turn preferably obtained by catalytic hydrogenation of dinitrotoluene. The overall process therefore particularly preferably comprises the following steps:
(A.I) catalytic hydrogenation of dinitrotoluene, optionally in the presence of a solvent, to obtain, optionally after removal of the solvent, a crude product fraction of tolylenediamine which contains not only isomers of tolylenediamine but also organic impurities.
(A.II) separation of water from the crude product fraction to obtain the meta-tolylenediamine-containing tolylenediamine isomer mixture;
(A.III) distillative workup of the meta-tolylenediamine-containing tolylenediamine isomer mixture to obtain the (first) meta-tolylenediamine product fraction and the tolylenediamine mixture.

The (first) meta-tolylenediamine product fraction obtained in step (A.III) is preferably phosgenated and a (first) meta-tolylene diisocyanate product is obtained by subsequent distillative workup.

The step of distillative workup, i.e. step (A.III) in the above-described particularly preferred embodiment, may in principle be performed according to any processes known from the prior art, for example according to a process such as is disclosed in the abovementioned patent U.S. Pat. No. 6,359,177 B1 (variant 1) or according to a process such as is disclosed in the abovementioned patent application EP 1 746 083 A1 (variant 2).

In a preferred embodiment of variant 1, which is based on FIG. 1 of the abovementioned patent U.S. Pat. No. 6,359,177 B1, the distillative workup comprises a distillation in a distillation column (in particular without a dividing wall) [apparatus A in said FIG. 1] with an evaporator arranged downstream thereof [apparatus B in said FIG. 1], wherein the tolylenediamine mixture is withdrawn from a (first) liquid bottoms stream from the evaporator [stream 5 in said FIG. 1] or a (second) liquid bottoms stream obtained by further workup thereof [stream 6 in said FIG. 1] and the (first) meta-tolylenediamine product fraction is obtained as a distillate fraction from the evaporator [stream 4 in said FIG. 1].

In another preferred embodiment of variant 1, which is based on FIG. 2 of the abovementioned patent U.S. Pat. No. 6,359,177 B1, the distillative workup comprises a distillation in a distillation column (in particular without a dividing wall) [apparatus A in said FIG. 2] with an evaporator arranged upstream thereof [apparatus B in said FIG. 2], wherein the tolylenediamine mixture is withdrawn from a (first) liquid bottoms stream from the evaporator [stream 50 in said FIG. 2] or a (second) liquid bottoms stream obtained by further workup thereof [stream 60 in said FIG. 2] and the (first) meta-tolylenediamine product fraction is obtained as a liquid bottoms fraction from the distillation column [stream 40 in said FIG. 2].

In a further preferred embodiment of variant 1, which is based on FIG. 3 of the abovementioned patent U.S. Pat. No. 6,359,177 B1, the distillative workup comprises a distillation in a distillation column (in particular without a dividing wall) [apparatus A in said FIG. 3], wherein the tolylenediamine mixture is withdrawn from a (first) liquid bottoms stream from the distillation column [stream 51 in said FIG. 3] or a (second) liquid bottoms stream obtained by further workup thereof [stream 61 in said FIG. 3] and the (first) meta-tolylenediamine product fraction is obtained as a gaseous bottoms fraction from the distillation column [corresponds to stream 41 in said FIG. 3 after condensation].

In a preferred embodiment of variant 2, which is thus based on the process disclosed in the abovementioned patent application EP 1 746 083 A1, the distillative workup comprises a distillation in a dividing wall column, wherein the tolylenediamine mixture is withdrawn from a (first) liquid bottoms stream from the dividing wall column [P4 in EP 1 746 083 A1] or from a (second) liquid bottoms stream obtained by further workup thereof and the (first) meta-tolylenediamine product fraction is obtained as a side draw fraction from the dividing wall column [P3 in EP 1 746 083 A1].

In a particular embodiment of variant 1 which is based on the abovementioned patent U.S. Pat. No. 6,359,177 B1 the tolylenediamine mixture is withdrawn from a (second) liquid bottoms stream obtained by further workup of the (first) liquid bottoms stream from the evaporator/the distillation column [i.e. stream 6, 60 or 61 in the drawings of U.S. Pat. No. 6,359,177 B1], wherein this further workup comprises a stripping [in the process according to US 6,359,177 B1 in apparatus C] and a concentrating [in the process according to U.S. Pat. No. 6,359,177 B1 in apparatus D] with addition of an ortho-tolylenediamine fraction distilled off in the distillation column [stream 3, 30 or 31 in the figures of U.S. Pat. No. 6,359,177 B1].

Suitable apparatuses for step (B) in this embodiment include in particular distillation apparatuses selected from the group consisting of kneader-dryers, fluidized bed dryers, roller-dryers, falling film evaporators, thin film evaporators, short path evaporators, screw dryers, natural circulation evaporators, forced circulation evaporators and decompression evaporators.

Suitable process conditions for step (B) in this embodiment include especially temperatures in the range from 60° C. to 240 C and pressures in the range from 0.1 mbar_{(abs.)} to 1013 mbar_{(abs.)}. Since in this embodiment the tolylenediamine distilled off in step (B) consists predominantly of ortho-tolylenediamine this is particularly suitable for further use as a starter for polyether polyol production, as a dye additive, as a starting material for the production of corrosion inhibitors or as a reagent in aminolysis reactions (in particular for cleavage urethane bonds in processes for recycling of polyurethane products).

In a further particular embodiment of the present invention, which may be combined both with variant 1 (which is based on the process described in the abovementioned patent U.S. Pat. No. 6,359,177 B1) and with variant 2 (which is based on the process described in the abovementioned patent application EP 1 746 083 A1) the tolylenediamine mixture is withdrawn from the (first) liquid bottoms stream from the evaporator [stream 5 or 50 in U.S. Pat. No. 6,359,177 B1] or the distillation column [stream 51 in U.S. Pat. No. 6,359,177 B1] or the dividing wall column [stream P4 in EP 1 746 083 A1]. Especially in this embodiment it can be particularly advantageous when in step (C), in addition to water, an amount of tolylenediamine smaller than that distilled off in step (B) is re-added, wherein (predominantly) meta TDA is distilled off in step (B) and (predominantly) ortho-TDA is subsequently re-added. This allows the meta-TDA which is particularly valuable for the majority of industrial applications to be obtained and the ortho-TDA which is less valuable for the majority of industrial applications to be used as solubilizer, the addition of which ensures that the mixture admixed with water is monophasic.

Suitable apparatuses for step (B) here likewise include evaporator apparatuses selected from the group consisting of kneader-dryers, fluidized bed dryers, roller-dryers, falling film evaporators, thin film evaporators, short path evaporators, screw dryers, natural circulation evaporators, forced circulation evaporators and decompression evaporators.

Suitable process conditions for step (B) in this embodiment include especially temperatures in the range from 98° C. to 270° C. and pressures in the range from 0.7 mbar_{(abs.)} to 730 mbar_{(abs.)}. Since in this embodiment the tolylenediamine distilled off in step (B) consists predominantly of meta-tolylenediamine this is particularly suitable for further use in the phosgenation to afford tolylene diisocyanate, wherein a (second) meta-tolylene diisocyanate product fraction (which may be obtained together with the first meta-tolylene diisocyanate product fraction) is obtained by subsequent distillative workup.

The pressure to be maintained in step (C) is independently of the precise configuration of step (A) preferably established by introduction of a gas selected from the group consisting of air, nitrogen, carbon dioxide, helium and argon. When a gas phase is present in the mixing space the pressure in the mixing space in the context of the invention is the pressure in this gas phase. A pressure control valve may also be employed in conjunction with the establishment of the pressure in the mixing introduction of a gas or as alternative an alternative thereto. A pressure control valve is employed especially when there is no gas phase present, i.e. two liquid streams are directly mixed with one another in step (C). The pressure established at said valve then corresponds to the pressure in the mixing space in the context of the invention. Irrespective of the precise configuration of step (C) the pressure in the mixing chamber is preferably 1.5 bar_{(abs.)} to 60 bar_{(abs.)}.

Step (C) preferably comprises incorporate insufficient water to ensure that the mass thereof corresponds to 10% to 100%, preferably 10% to 80%, of the mass of the tolylenediamine distilled off in step (B). It has further proven advantageous to adjust the temperature of the water to be incorporated in step (C) to 10° C. to 100° C., preferably 20° C. to 90° C. The following temperature ranges are particularly preferred:
10° C. to 40° C., especially 20° C. to 40° C. (so that fresh water or low-temperature condensate is a suitable water source)
80° C. to 100° C., especially 80° C. to 90° C. (so that high-temperature condensate is a suitable water source).

It is preferable when the mixture admixed with water obtained in step (C) is cooled to a temperature in the range from 20° C. to 100° C., preferably 20° C. to 90° C., and decompressed, especially to ambient pressure, in a step (D.I) as preparation for further use thereof, namely a use as material (i.e. as a starting material for obtaining other products) or a thermal use (i.e. as fuel). Cooling may be effected by active cooling or simply by allowing to cool to the desired temperature, wherein the latter is preferred. The energy liberated in step (D.I) may advantageously be supplied (energy integration) to various energy consumers (for example endothermic processes). The mixture treated in this way may then be supplied to a further use as material or thermal use in a step (E). The process according to the invention has the advantage that this further use need not immediately follow step (D.I) because even after cooling and decompression the mixtures admixed with water obtained in step (C) are storage stable for a relatively long time at a temperature in the range from ambient temperature to the temperature to which they were cooled in step (D.I). This further allows transport of the mixture to a production site which is spatially separate from the production side of steps (A) to (D). The process according to the invention especially allows storage and/or transport to altogether account for a period of 1 h to 720 h, preferably 24 h to 480 h, particularly preferably 48 h to 360 h. The further use as material of the mixtures in step (E) is preferably selected from the use as fuel and as a feedstock in a pyrolysis for recovery of benzene, toluene and/or xylene.

According to the circumstances the mixture admixed with water obtained in step (C) may also be supplied (immediately) after step (C) to a thermal utilization, i.e. a use as fuel (step (D.II). This is possible if there is an incinerator at the production site of steps (A) to (C).

Cooling and decompression may then be omitted. It goes without saying that unintentional cooling and decompression during pumping through pipe conduits to the incineration furnace is possible and does not depart from the scope of this embodiment of the invention.

The process according to the invention allows recovery of valuable TDA from TDA residue mixtures without the remaining residue becoming impracticable to process due to excessive viscosity. Without wishing to be bound to a particular theory it is thought that as a result of the inventive procedure of distillative removal of TDA followed by incorporation of water at elevated temperature and elevated pressure the state of an emulsion is “frozen” and the remaining residue therefore remains low over industrially relevant timeframes.

### EXAMPLES

### Example 1

### Discontinuous Mixing at Superatmospheric Pressure (Inventive)

A heatable stirred tank was filled with a mixture of ortho-TDA and TDA residue (mass ratio 1:1.5) that had been preheated to 80° C. using a water bath (step (A)) followed by heating of the mixture to 140° C. using thermal oil while simultaneously establishing a reduced pressure of 2 mbar_{(abs.)} in the stirred tank. 70% of the mass of the ortho-TDA was then distilled off (Step (B)), condensed in an external condenser and collected. Subsequently, the tank contents having a temperature of 140° C. (containing 17% by mass of ortho TDA) were pressurized with nitrogen at a pressure of 4 bar_{(abs.)} and water (33% of the mass of the ortho-TDA distilled off) having a temperature of 80° C. at a pressure of 4.5 bar_{(abs.)} was injected into the container in stepwise fashion and mixed until a homogeneous phase was formed (step (C)). This homogeneous phase contained water in a mass fraction of 11.5% based on its total mass. This resulted in a mixing temperature of 135° C. was . After mixing, the vessel was cooled to 80° C. and decompressed to the atmosphere and the liquid mixture was discharged (step (D.I)). Compared to the starting mixture the obtained residue mixture had low-viscosity properties (144 mPa s instead of 326 mPa s) and remained stable over at least 4 weeks (even upon further cooling to room temperature).

### Example 2

### Discontinuous Mixing at Superatmospheric Pressure (Inventive)

A heatable stirred tank was filled with a mixture of ortho-TDA and TDA residue (mass ratio 1:1.5) that had been preheated to 80° C. using a water bath (step (A)) followed by heating of the mixture to 140° C. using thermal oil while simultaneously establishing a reduced pressure of 2 mbar_{(abs.)} in the stirred tank. Subsequently 25% of the mass of the ortho-TDA was distilled off (Step (B)), condensed in an external condenser and collected. Subsequently, the tank contents having a temperature of 140° C. (containing 33% by mass of ortho TDA) were pressurized with nitrogen at a pressure of 4 bar_{(abs.)} and water (100% of the mass of the ortho-TDA distilled off) having a temperature of 20° C. at a pressure of 4.5 bar_{(abs.)} was injected into the container all at once and mixed until a homogeneous phase was formed (step (C)). This homogeneous phase contained water in a mass fraction of 15% based on its total mass. This resulted in a mixing temperature of 119° C. After mixing, the vessel was cooled to 80° C. and decompressed to the atmosphere and the liquid mixture was discharged (step (D.I)). Compared to the starting mixture the obtained residue mixture had low-viscosity properties (35 mPa s instead of 326 mPa s) and remained stable over at least 4 weeks (even upon further cooling to room temperature).

### Example 3

### Continuous Mixing at Superatmospheric Pressure (Simulation; Inventive)

The process comprises mixing a continuously obtained residue-rich waste stream (84% by mass of TDA residue and 16% by mass of ortho-TDA) obtained by distillative removal of TDA (step (B)) from a TDA mixture (containing 60% TDA residue and 40% ortho-TDA;

(step (A)) with water (step (C)) at superatmospheric pressure.

The process product depleted in TDA continuously obtained at 220° C. in the distillative removal of TDA in step (B) (containing 84% TDA residue and 16% ortho-TDA) is initially pre-cooled to 180° C. in a heat exchanger for performance of step (C). This is followed by addition of water having a temperature of 20° C. at a pressure of 6 bar_{(abs.)} to obtain a mixture admixed with water having a temperature of 155° C., in which water is in the liquid state. The hot process product depleted in TDA may likewise be pre-cooled from 220° C. to 165° C. and an addition of water having a temperature of 90° C. at a pressure of 6 bar_{(abs.)} performed to obtain an analogous mixture admixed with water having a temperature of 155° C. The mixture admixed with water is subsequently pumped through static mixers and then further decompressed to ambient pressure and cooled to a temperature in the range from 20° C. to 100° C. (step (D.I)), transported, stored and supplied to a further use as material (step (E)). The mixture admixed with water may also be incinerated (step (D.II)).

The example shows that the process according to the invention makes it possible to reduce the TDA content (here ortho-TDA) in the mixture admixed with water to 16% by mass while the obtained mixture admixed with water nevertheless remains processable, in particular pumpable at low temperatures. According to the teaching of WO 2018/140461 A1 the content of TDA in the TDA residue composition to be admixed with water is not less than 40% by mass.

### Example 4

### Discontinuous Mixing at Superatmospheric Pressure (Comparative—Excessively Low Temperature In Step (C))

A heatable stirred tank was filled with a mixture of ortho-TDA and TDA residue (mass ratio 1:1.5) that had been preheated to 80° C. using a water bath (step (A)) followed by heating of the mixture to 120° C. using thermal oil while simultaneously establishing a reduced pressure of 2 mbar_{(abs.)} in the stirred tank. 70% of the mass of the ortho-TDA was then distilled off (Step (B)), condensed in an external condenser and collected. Subsequently, the tank contents having a temperature of 120° C. (containing 17% by mass of ortho TDA) were pressurized with nitrogen at a pressure of 4 bar_{(abs.)} and water (33% of the mass of the ortho-TDA distilled off) having a temperature of 25° C. at a pressure of 4.5 bar_{(abs.)} was injected into the container all at once and mixed (step (C)). The obtained mixture contained water in a mass fraction of 11.5% based on its total mass. The mixing temperature fell below 100° C. and the mixture was biphasic as a result. The temperature of the mixture was then increased to 140° C. with constant stirring. However, the mixture remained biphasic despite prolonged stirring.

1. A process for working upworkup of a tolylenediamine mixture containing tolylenediamine and compounds having boiling points higher than 2,4-tolylenediamine at 1013 mbar_{(abs.)}, comprising the steps of:
(A) providing a tolylenediamine mixture containing (1) tolylenediamine in an amount of 5% by mass to 80% by mass and (2) compounds having boiling points higher than 2,4-tolylenediamine at 1013 mbar_{(abs.)} in an amount of 20% by mass to 95% by mass, based on total mass of the tolylenediamine mixture;
(B) distilling the tolylenediamine mixture to remove tolylenediamine therefrom and to obtain a liquid process product-depleted in tolylenediaminc containing (1) 0% by mass to 38% by mass of tolylenediamine, and (2) 62% by mass to 100% by mass of compounds having boiling points higher than 2,4-tolylenediamine at 1013 mbar_{(abs.)} (high boilers), based on total mass of the liquid process product;
C) incorporating water into the process product depleted in tolylenediamine in a mixing space to obtain a mixture admixed with water, wherein the temperature and amount of the water incorporated and the temperature and amount of the process product depleted in tolylenediamine are matched to one another to provide a temperature of the mixture admixed with water of 110° C. to 250° C. and the mixture admixed with water is monophasic, wherein a pressure which is greater than or equal to the water vapor partial pressure at the resulting temperature is established in the mixing space.

2. The process as claimed in claim 1 wherein in step (C) the pressure in the mixing space is established by introducing a gas selected from the group consisting of air, nitrogen, carbon dioxide, helium and argon and/or via a pressure control valve.

3. The process as claimed in claim 1, wherein the mass of the water incorporated in step (C) is 10% to 100% of the mass of the tolylenediamine distilled off in step (B).

4. The process as claimed in claim 1, further comprising:
(D.I) preparing the mixture admixed with water for a use as material or for a thermal use comprising decompressing and cooling the mixture admixed with water to a temperature of 20° C. to 100° C. to obtain a decompressed and cooled mixture admixed with water; and
(E) supplying the decompressed and cooled mixture admixed with water obtained in step (D) to a use as material or thermal use.

5. The process as claimed in claim 4, wherein the decompressed and cooled mixture admixed with water is, prior to its use as material or thermal use in step (E), stored and/or transported at a temperature of ambient temperature to the temperature to which it is cooled in step (D.I), wherein the storing and transporting altogether account for a period of 1 hour to 720 hours.

6. The process as claimed in claim 1, further comprising:
(D.II) using the mixture admixed with water as a fuel.

7. The process as claimed in claim 1, wherein the tolylenediamine mixture in step (A) is provided by distillative workup of a meta-tolylenediamine-containing tolylenediamine isomer mixture, wherein a meta-tolylenediamine product fraction is obtained in addition to the tolylenediamine mixture.

8. The process as claimed in claim 7,
wherein the distillative workup (1) comprises a distillation in a distillation column with an evaporator arranged downstream thereof, wherein the tolylenediamine mixture is withdrawn from a liquid bottoms stream from the evaporator or from a liquid bottoms stream obtained by further workup thereof and the meta-tolylenediamine product fraction is obtained as a distillate fraction from the evaporator;
or wherein the distillative workup (2) comprises a distillation in a distillation column with an evaporator arranged upstream thereof, wherein the tolylenediamine mixture is withdrawn from a liquid bottoms stream from the evaporator or from a liquid bottoms stream obtained by further workup thereof and the meta-tolylenediamine product fraction is obtained as a liquid bottoms fraction from the distillation column;
or wherein the distillative workup (3) comprises a distillation in a distillation column, wherein the tolylenediamine mixture is withdrawn from a liquid bottoms stream from the distillation column or from a liquid bottoms stream obtained by further workup thereof and the meta-tolylenediamine product fraction is obtained as a gaseous bottoms fraction from the distillation column;
or wherein the distillative workup (4) comprises a distillation in a dividing wall column, wherein the tolylenediamine mixture is withdrawn from a liquid bottoms stream from the dividing wall column or from a liquid bottoms stream obtained by further workup thereof and the meta-tolylenediamine product fraction is obtained as a side draw fraction from the dividing wall column.

9. The process as claimed in claim 8, comprising one or more of the variants (1) to (3), but not variant (4), wherein the tolylenediamine mixture is withdrawn from a liquid bottoms stream obtained by further workup of the liquid bottoms stream from the evaporator or the distillation column, wherein the further workup comprises a stripping and a concentrating with addition of an ortho-tolylenediamine fraction distilled off in the distillation column.

10. The process as claimed in claim 9, wherein the toluenediamine distilled off in step (B) is used as a starter for polyether polyol production, as a dye additive, as a starting material for the production of a corrosion inhibitor or in an aminolysis reaction.

11. The process as claimed in claim 8, wherein the tolylenediamine mixture is withdrawn from the liquid bottoms stream from the evaporator or the distillation column or the dividing wall column.

12. The process as claimed in claim 11, wherein, in step (C), in addition to water, an amount of tolylenediamine smaller than that distilled off in step (B) is re-added, wherein the ratio of the tolylenediamine isomers to one another in the tolylenediamine distilled off on the one hand and in the tolylenediamine re-added on the other hand are selected such that molar ratio of ortho-tolylenediamine to meta-tolylenediamine in the mixture admixed with water is increased relative to the process product depleted in tolylenediamine.

13. The process as claimed in claim 11, wherein the tolylenediamine distilled off in step (B) is phosgenated to produce tolylene diisocyanate, and wherein a meta-tolylene diisocyanate product fraction is obtained by subsequent distillative workup.

14. The process as claimed in claim 7, wherein step (A) comprises:
(AI) catalytic hydrogenation of dinitrotoluene, optionally in the presence of a solvent, to obtain, optionally after removal of the solvent, a crude product fraction of tolylenediamine which contains not only isomers of tolylenediamine but also organic impurities;
(A.II) separation of water from the crude product fraction to obtain the meta-tolylenediamine-containing tolylenediamine isomer mixture; and
(A.III) distillative workup of the meta-tolylenediamine-containing tolylenediamine isomer mixture to obtain the meta-tolylenediamine product fraction and the tolylenediamine mixture.
